(19) United States
(12) Patent Application Publication
(10) Pub. No.: US 2010/0076559 A1
(43) Pub. Date: Mar. 25, 2010

(54) Composite telescoping anterior interbody spinal implant

(75) Inventors: Charanpreet S. Bagga, Phoenixville, PA (US); Peter F. Ullrich, JR., Neenah, WI (US)

Correspondence Address: STRADLEY RONON STEVENS & YOUNG, LLP, 30 VALLEY STREAM PARKWAY, GREAT VALLEY CORPORATE CENTER, MALVERN, PA, 19355-1481, US

(73) Assignee: Titan Spine, LLC

(21) Appl. No.: 12/151,167

(22) Filed: May 5, 2008

Related U.S. Application Data
(60) Provisional application No. 60/927,770, filed on May 4, 2007.

(51) Int. Cl. A61F 2/44

(52) U.S. Cl. 623/17.16

(57) ABSTRACT

A composite telescoping interbody spinal implant and method of using the implant. The implant includes a cage formed of metal, a metal alloy, or both. The cage is able to change size following manufacture, and has a top plate with a plurality of posts and a bottom plate with a corresponding plurality of columns. The posts telescopically engage the columns upon assembly of the top plate with the bottom plate. The posts extend partially outside the columns when the top plate is in a raised first position with respect to the bottom plate; the posts and columns are fully engaged when the top plate is in a second position closest to the bottom plate. The implant also includes a non-metallic body inserted between the top plate and the bottom plate and defining the adjustable height of the implant.

### RELATED APPLICATION

This application claims the benefit of priority to U.S. Provisional Patent Application Ser. No. 60/927,770, filed on May 4, 2007, the contents of which are incorporated in this document by reference.

### TECHNICAL FIELD

The present invention relates generally to interbody spinal implants and methods of using such implants and, more particularly, to a composite telescoping interbody spinal implant.

### BACKGROUND OF THE INVENTION

In the simplest terms, the spine is a column made of vertebrae and discs. The vertebrae provide the support and structure of the spine while the spinal discs, located between the vertebrae, act as cushions or “shock absorbers.” The discs also contribute to the flexibility and motion of the spinal column. Over time, the discs may become diseased or infected, may develop deformities such as tears or cracks, or may simply lose structural integrity (e.g., the discs may bulge or flatten). Impaired discs can affect the anatomical functions of the vertebrae, due to the resultant lack of proper biomechanical support, and are often associated with chronic back pain.

Several surgical techniques have been developed to address such spinal defects as disc degeneration and deformity. Spinal fusion has become a recognized surgical procedure for mitigating back pain by restoring biomechanical and anatomical integrity to the spine. Spinal fusion techniques involve the removal, or partial removal, of at least one intervertebral disc and preparation of the disc space for receiving an implant by shaping the exposed vertebral endplates. An implant is then inserted between the opposing endplates.

Spinal fusion procedures can be achieved using a posterior or an anterior approach. Anterior interbody fusion procedures generally have the advantages of reduced operative times and reduced blood loss. Further, anterior procedures do not interfere with the posterior anatomic structure of the lumbar spine. Anterior procedures also minimize scarring within the spinal canal while still achieving improved fusion rates, which is advantageous from a structural and biomechanical perspective. These generally preferred anterior procedures are particularly advantageous in providing improved access to the disc space, and thus correspondingly better endplate preparation.

Several interbody implant systems have been introduced to facilitate interbody fusion. Traditional threaded implants involve at least two cylindrical bodies, each typically packed with bone graft material, surgically placed on opposite sides of the mid-sagittal plane through pre-tapped holes within the intervertebral disc space. This location is not the preferable seating position for an implant system, however, because only a relatively small portion of the vertebral endplate is contacted by these cylindrical implants. Accordingly, these implant bodies will likely contact the softer cancellous bone rather than the stronger cortical bone, or apophyseal rim, of the vertebral endplate. The seating of these threaded cylindrical implants may also compromise biomechanical integrity by reducing the area in which to distribute mechanical forces, thus increasing the apparent stress experienced by both the implant and vertebrae. Still further, a substantial risk of implant subsidence (defined as sinking or settling) into the softer cancellous bone of the vertebral body may arise from such improper seating.

In contrast, open ring-shaped cage implant systems are generally shaped to mimic the anatomical contour of the vertebral body. Traditional ring-shaped cages are generally comprised of allograft bone material, however, harvested from the human femur. Such allograft bone material restricts the usable size and shape of the resultant implant. For example, many of these femoral ring-shaped cages generally have a medial-lateral width of less than 25 mm. Therefore, these cages may not be of a sufficient size to contact the strong cortical bone, or apophyseal rim, of the vertebral endplate. These size-limited implant systems may also poorly accommodate related instrumentation such as drivers, reamers, distractors, and the like. For example, these implant systems may lack sufficient structural integrity to withstand repeated impact and may fracture during implantation. Still further, other traditional non-allograft ring-shaped cage systems may be size-limited due to varied and complex supplemental implant instrumentation which may obstruct the disc space while requiring greater exposure of the operating space. These supplemental implant instrumentation systems also generally increase the instrument load upon the surgeon.

The surgical procedure corresponding to an implant system should preserve as much vertebral endplate bone surface as possible by minimizing the amount of bone removed. This vertebral endplate bone surface, or subchondral bone, is generally much stronger than the underlying cancellous bone. Preservation of the endplate bone stock ensures biomechanical integrity of the endplates and minimizes the risk of implant subsidence. Thus, proper interbody implant design should provide for optimal seating of the implant while utilizing the maximum amount of available supporting vertebral bone stock.

Traditional interbody spinal implants generally do not seat properly on the preferred structural bone located near the apophyseal rim of the vertebral body, which is primarily composed of preferred dense subchondral bone. Accordingly, there is a need in the art for interbody spinal implants which better utilize the structurally supportive bone of the apophyseal rim.

In summary, at least ten, separate challenges can be identified as inherent in traditional anterior spinal fusion devices. Such challenges include: (1) end-plate preparation; (2) implant difficulty; (3) materials of construction; (4) implant expulsion; (5) implant subsidence; (6) insufficient room for bone graft; (7) stress shielding; (8) lack of implant incorporation with vertebral bone; (9) limitations on radiographic visualization; and (10) cost of manufacture and inventory. Each of these challenges is addressed in turn.

1. End-Plate Preparation

There are three traditional end-plate preparation methods. The first is aggressive end-plate removal with box-chisel types of tools to create a nice match of end-plate geometry with implant geometry. In the process of aggressive end-plate removal, however, the end-plates are typically destroyed. Such destruction means that the load-bearing implant is pressed against soft cancellous bone and the implant tends to subside.

The second traditional end-plate preparation method preserves the end-plates by just removing cartilage with curettes. The end-plates are concave; hence, if a flat implant is used, the implant is not very stable. Even if a convex implant is used, it is very difficult to match the implant geometry with the end-plate geometry, as the end-plate geometry varies from patient-to-patient and on the extent of disease.

The third traditional end-plate preparation method uses threaded fusion cages. The cages are implanted by reaming out corresponding threads in the end-plates. This method also violates the structure.

2. Implant Difficulty

Traditional anterior spinal fusion devices can also be difficult to implant. Some traditional implants with teeth have sharp edges. These edges can bind to the surrounding soft tissue during implantation, creating surgical challenges.

Typically, secondary instrumentation is used to keep the disc space distracted during implantation. The use of such instrumentation means that the exposure needs to be large enough to accommodate the instrumentation. If there is a restriction on the exposure size, then the maximum size of the implant available for use is correspondingly limited. The need for secondary instrumentation for distraction during implantation also adds an additional step or two in surgery. Still further, secondary instrumentation may sometimes over-distract the annulus, reducing the ability of the annulus to compress a relatively undersized implant. The compression provided by the annulus on the implant is important to maintain the initial stability of the implant.

For anterior spinal surgery, there are traditionally three trajectories of implants: anterior, antero-lateral, and lateral. Each approach has its advantages and drawbacks. Sometimes the choice of the approach is dictated by surgeon preference, and sometimes it is dictated by patient anatomy and biomechanics. A typical traditional implant has design features to accommodate only one or two of these approaches in a single implant, restricting intra-operative flexibility.

3. Materials of Construction

Other challenges raised by traditional devices find their source in the conventional materials of construction. Typical devices are made of PEEK or cadaver bone. Materials such as PEEK or cadaver bone do not have the structural strength to withstand impact loads required during implantation and may fracture during implantation.

PEEK is an abbreviation for polyetherether-ketone, a high-performance engineering thermoplastic with excellent chemical and fatigue resistance plus thermal stability. With a maximum continuous working temperature of 480° F., PEEK offers superior mechanical properties. Superior chemical resistance has allowed PEEK to work effectively as a metal replacement in harsh environments. PEEK grades offer chemical and water resistance similar to PPS (polyphenylene sulfide), but can operate at higher temperatures. PEEK materials are inert to all common solvents and resist a wide range of organic and inorganic liquids. Thus, for hostile environments, PEEK is a high-strength alternative to fluoropolymers.

The use of cadaver bone has several drawbacks. The shapes and sizes of the implants are restricted by the bone from which the implant is machined. Cadaver bone carries with it the risk of disease transmission and raises shelf-life and storage issues. In addition, there is a limited supply of donor bone and, even when available, cadaver bone inherently offers inconsistent properties due to its variability. Finally, as mentioned above, cadaver bone has insufficient mechanical strength for clinical application.

4. Implant Expulsion

Traditional implants can migrate and expel out of the disc space, following the path through which the implant was inserted. Typical implants are either “threaded” into place, or have “teeth” which are designed to prevent expulsion. Both options can create localized stress risers in the end-plates, increasing the chances of subsidence. The challenge of preventing implant expulsion is especially acute for PEEK implants, because the material texture of PEEK is very smooth and “slippery.”

5. Implant Subsidence

Subsidence of the implant is a complex issue and has been attributed to many factors. Some of these factors include aggressive removal of the end-plate; an implant stiffness significantly greater than the vertebral bone; smaller sized implants which tend to seat in the center of the disc space, against the weakest region of the end-plates; and implants with sharp edges which can cause localized stress fractures in the end-plates at the point of contact. The most common solution to the problem of subsidence is to choose a less stiff implant material. This is why PEEK and cadaver bone have become the most common materials for spinal fusion implants. PEEK is softer than cortical bone, but harder than cancellous bone.

6. Insufficient Room for Bone Graft

Cadaver bone implants are restricted in their size by the bone from which they are machined. Their wall thickness also has to be great to create sufficient structural integrity for their desired clinical application. These design restrictions do not leave much room for filling the bone graft material into cortical bone implants. The exposure-driven limitations on implant size narrow the room left inside the implant geometry for bone grafting even for metal implants. Such room is further reduced in the case of PEEK implants because their wall thickness needs to be greater as compared to metal implants due to structural strength needs.

7. Stress Shielding

For fusion to occur, the bone graft packed inside the implant needs to be loaded mechanically. Typically, however, the stiffness of the implant material is much greater than the adjacent vertebral bone and takes up a majority of the mechanical loads, “shielding” the bone graft material from becoming mechanically loaded. The most common solution is to choose a less stiff implant material. Again, this is why PEEK and cadaver bone have become the most common materials for spinal fusion implants. As noted above, although harder than cancellous bone, PEEK is softer than cortical bone.

8. Lack of Implant Incorporation with Vertebral Bone

In most cases, the typical fusion implant is not able to incorporate with the vertebral bone, even years after implantation. Such inability persists despite the use of a variety of different materials used to construct the implants. There is a perception that cadaver bone is resorbable and will be replaced by new bone once it resorbs. Hedrocel is a composite material composed of carbon and tantalum, an inert metal, that has been used as a material for spinal fusion implants. Hedrocel is designed to allow bone in-growth into the implant. In contrast, PEEK has been reported to become surrounded by fibrous tissue which precludes it from incorporating with surrounding bone. There have also been reports of the development of new bio-active materials which can incorporate into bone. The application of such bio-active materials has been limited, however, for several reasons, including biocompatibility, structural strength, and lack of regulatory approval.

9. Limitations on Radiographic Visualization

For implants made out of metal, the metal prevents adequate radiographic visualization of the bone graft. Hence it is difficult to assess fusion, if it is to take place. PEEK is radiolucent. Traditional implants made of PEEK need to have radiographic markers embedded into the implants so that implant position can be tracked on an X-ray. Cadaver bone has some radiopacity and does not interfere with radiographic assessment as much as metal implants.

10. Cost of Manufacture and Inventory

The requirements of spinal surgery dictate that manufacturers provide implants of various foot-prints, and several heights in each foot-print. This requirement means that the manufacturer needs to carry a significant amount of inventory of implants. Because there are so many different sizes of implants, there are setup costs involved in the manufacture of each different size. The result is increased implant costs, which the manufacturers pass along to the end users by charging high prices for spinal fusion implants.

### BRIEF SUMMARY OF THE INVENTION

The present invention is directed to interbody spinal implants and to methods of using such implants. Although they can be implanted from a variety of vantages, including anterior, antero-lateral, and lateral implantation, the interbody spinal implants are particularly suited for placement using an anterior surgical approach. Certain embodiments of the present invention provide an anatomically shaped spinal implant for improved seating in the disc space, particularly in the medial-lateral aspect of the disc space, and improved utilization of the vertebral apophyseal rim. Certain embodiments of the present invention further have a highly radiused posterior portion and sides which allow for ease of implantation. Thus, the posterior portion may have a generally blunt nosed profile. Certain embodiments also allow for improved visualization of the disc space during surgical procedures while minimizing exposure of the operating space. Certain aspects of the invention reduce the need for additional instrumentation—such as chisels, reamers, or other tools—to prepare the vertebral endplate, thus minimizing the instrument load upon the surgeon.

Certain embodiments of the interbody implant are substantially hollow and have a generally oval-shaped transverse cross-sectional area. Substantially hollow, as used in this document, means at least about 33% of the interior volume of the interbody spinal implant is vacant. Further embodiments of the present invention include a body having a top surface, a bottom surface, opposing lateral sides, and opposing anterior and posterior portions. The implant includes at least one aperture that extends the entire height of the body. Thus, the aperture extends from the top surface to the bottom surface. The implant may further include at least one aperture that extends the entire transverse length of the implant body.

Still further, the substantially hollow portion may be filled with cancellous autograft bone, allograft bone, demineralized bone matrix (DBM), porous synthetic bone graft substitute, bone morphogenic protein (BMP), or combinations of those materials. The implant further includes a roughened surface topography on at least a portion of its top surface, its bottom surface, or both surfaces. The anterior portion, or trailing edge, of the implant is preferably generally greater in height than the opposing posterior portion, or leading edge. In other words, the trailing edge is taller than the leading edge. The posterior portion and lateral sides may also be generally smooth and highly radiused, thus allowing for easier implantation into the disc space. Thus, the posterior portion may have a blunt nosed profile. The anterior portion of the implant may preferably be configured to engage a delivery device, a driver, or other surgical tools. The anterior portion may also be substantially flat.

According to certain embodiments, the present invention provides a composite telescoping interbody spinal implant and a method of using that implant. The implant includes a cage formed of metal, a metal alloy, or both. The cage is able to change size following manufacture, and has a top plate with a plurality of posts and a bottom plate with a corresponding plurality of columns. The posts telescopically engage the columns upon assembly of the top plate with the bottom plate. The posts extend partially outside the columns when the top plate is in a raised first position with respect to the bottom plate; the posts and columns are fully engaged when the top plate is in a second position closest to the bottom plate. The implant also includes a non-metallic body inserted between the top plate and the bottom plate and defining the adjustable height of the implant.

It is to be understood that both the foregoing general description and the following detailed description are exemplary, but are not restrictive, of the invention.

### BRIEF DESCRIPTION OF THE DRAWING

The invention is best understood from the following detailed description when read in connection with the accompanying drawing. It is emphasized that, according to common practice, the various features of the drawing are not to scale. On the contrary, the dimensions of the various features are arbitrarily expanded or reduced for clarity. Included in the drawing are the following figures:

FIG. 1 shows a perspective view of a first embodiment of the interbody spinal implant having a generally oval shape and roughened surface topography on the top surface;

FIG. 2 depicts a top view of the first embodiment of the interbody spinal implant;

FIG. 3 depicts an anterior view of the first embodiment of the interbody spinal implant;

FIG. 4 depicts a posterior view of the first embodiment of the interbody spinal implant;

FIG. 5A depicts a first post-operative radiograph showing visualization of an embodiment of the interbody spinal implant;

FIG. 5B depicts a second post-operative radiograph showing visualization of an embodiment of the interbody spinal implant;

FIG. 5C depicts a third post-operative radiograph showing visualization of an embodiment of the interbody spinal implant;

FIG. 6 shows an exemplary surgical tool (implant holder) to be used with certain embodiments of the interbody spinal implant;

FIG. 7 shows an exemplary distractor used during certain methods of implantation;

FIG. 8 shows an exemplary rasp used during certain methods of implantation;

FIG. 9A illustrates the top plate of the cage forming another embodiment of the interbody spinal implant according to the present invention;

FIG. 9B illustrates the bottom plate of the cage forming another embodiment of the interbody spinal implant according to the present invention;

FIG. 9C illustrates the top plate of the cage formed as two, separate sections to create yet another embodiment of the interbody spinal implant according to the present invention;

FIG. 9D illustrates the bottom plate of the cage formed as two, separate sections to create yet another embodiment, in combination with the top plate illustrated in FIG. 9C, of the interbody spinal implant according to the present invention;

FIG. 10 shows the top plate of FIG. 9A and the bottom plate of FIG. 9B in their assembled position to form the cage;

FIG. 11 depicts an anterior view of the assembled cage shown in FIG. 10 with the top plate fully seated on the bottom plate;

FIG. 12 depicts another anterior view of the assembled cage shown in FIG. 10, illustrating the telescopic feature of the present invention;

FIG. 13 is the same anterior view of the assembled cage shown in FIG. 12, but depicts the interior channels that extend vertically within each of the female columns;

FIG. 14 is a lateral side view of the assembled cage shown in FIG. 13;

FIG. 15 is a perspective view of the assembled cage shown in FIG. 13;

FIG. 16 is a perspective view of a composite interbody spinal implant showing the cage, including the top plate and the bottom plate in their assembled position, combined with the body;

FIG. 17A is a top view of the top plate of yet another embodiment of the composite interbody spinal implant according to the present invention, including four struts;

FIG. 17B depicts an anterior view of the embodiment of the interbody spinal implant shown in FIG. 17A;

FIG. 17C depicts a side view of the embodiment of the interbody spinal implant shown in FIGS. 17A and 17B;

FIG. 17D depicts a perspective view of the embodiment of the interbody spinal implant shown in FIGS. 17A, 17B, and 17C;

FIG. 18 is a perspective view of the top plate of yet another embodiment of the composite interbody spinal implant according to the present invention, including three struts;

FIG. 19A is a perspective view, from a first lateral-posterior vantage, of yet another embodiment of the composite interbody spinal implant according to the present invention, including struts of different geometries;

FIG. 19B is a perspective view, from a second lateral-posterior vantage, of the embodiment of the interbody spinal implant shown in FIG. 19A;

FIG. 19C is a perspective view, from a lateral-anterior vantage, of the embodiment of the interbody spinal implant shown in FIGS. 19A and 19B;

FIG. 19D is the same perspective view of the embodiment of the interbody spinal implant shown in FIG. 19C, illustrating the posts of the top plate as inserted in the columns of the bottom plate;

FIG. 20 is a perspective view of the cage forming still another embodiment of the interbody spinal implant according to the present invention, illustrating a cage having four posts on the top plate and four corresponding columns on the bottom plate and eliminating the front face of the top plate; and

FIG. 21 is a perspective view of the cage forming a further embodiment of the interbody spinal implant according to the present invention, illustrating a cage having four posts on abbreviated top plate sections and four corresponding columns on abbreviated bottom plate sections and eliminating much of the top and bottom plates.

### DETAILED DESCRIPTION OF THE INVENTION

Certain embodiments of the present invention may be especially suited for placement between adjacent human vertebral bodies. The implants of the present invention may be used in procedures such as cervical fusion and Anterior Lumbar Interbody Fusion (ALIF). Certain embodiments do not extend beyond the outer dimensions of the vertebral bodies.

The ability to achieve spinal fusion is directly related to the available vascular contact area over which fusion is desired, the quality and quantity of the fusion mass, and the stability of the interbody spinal implant. Interbody spinal implants, as now taught, allow for improved seating over the apophyseal rim of the vertebral body. Still further, interbody spinal implants, as now taught, better utilize this vital surface area over which fusion may occur and may better bear the considerable biomechanical loads presented through the spinal column with minimal interference with other anatomical or neurological spinal structures. Even further, interbody spinal implants, according to certain aspects of the present invention, allow for improved visualization of implant seating and fusion assessment. Interbody spinal implants, as now taught, may also facilitate osteointegration with the surrounding living bone.

Anterior interboody spinal implants in accordance with certain aspects of the present invention can be preferably made of a durable material such as stainless steel, stainless steel alloy, titanium, or titanium alloy, but can also be made of other durable materials such as, but not limited to, polymeric, ceramic, and composite materials. For example, certain embodiments of the present invention may be comprised of a biocompatible, polymeric matrix reinforced with bioactive fillers, fibers, or both. Certain embodiments of the present invention may be comprised of urethane dimethacrylate (DUDMA)/tri-ethylene glycol dimethacrylate (TEDGMA) blended resin and a plurality of fillers and fibers including bioactive fillers and E-glass fibers. Durable materials may also consist of any number of pure metals, metal alloys, or both. Titanium and its alloys are generally preferred for certain embodiments of the present invention due to their acceptable, and desirable, strength and biocompatibility. In this manner, certain embodiments of the present interbody spinal implant may have improved structural integrity and may better resist fracture during implantation by impact. Interbody spinal implants, as now taught, may therefore be used as a distractor during implantation.

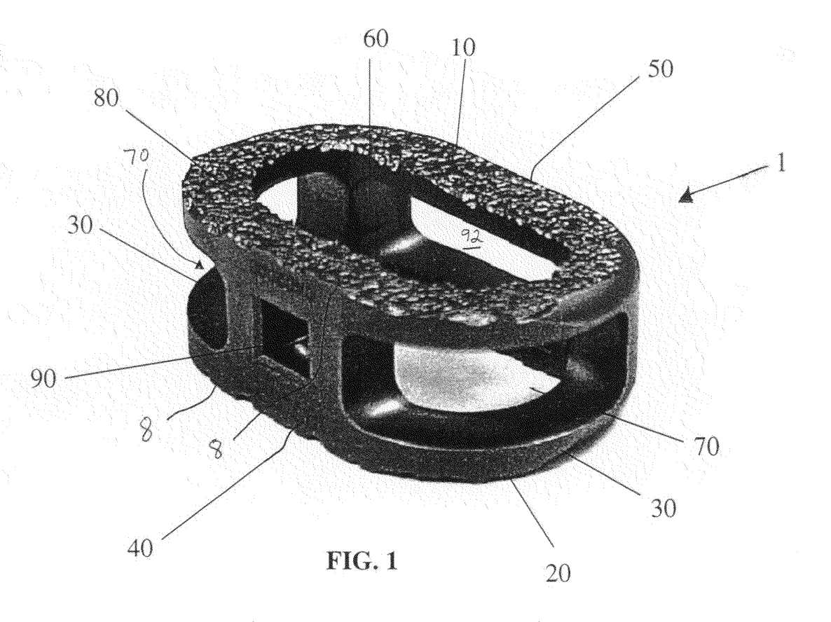

Referring now to the drawing, in which like reference numbers refer to like elements throughout the various figures that comprise the drawing, FIG. 1 shows a perspective view of a first embodiment of the interbody spinal implant 1. The interbody spinal implant 1 includes a body having a top surface 10, a bottom surface 20, opposing lateral sides 30, and opposing anterior 40 and posterior 50 portions. One or both of the top surface 10 and the bottom surface 20 has a roughened topography 80. Distinguish the roughened topography 80, however, from the disadvantageous teeth provided on the surfaces of some conventional devices.

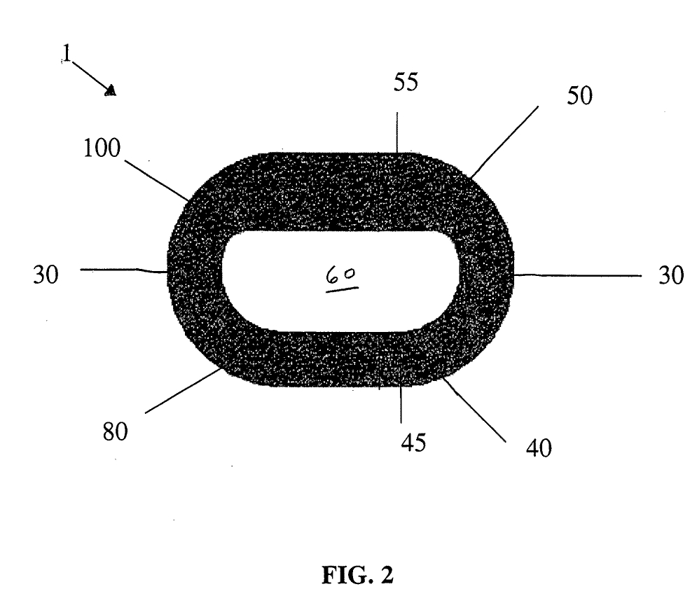

Certain embodiments of the interbody spinal implant 1 are substantially hollow and have a generally oval-shaped transverse cross-sectional area with smooth, rounded, or both smooth and rounded lateral sides and posterior-lateral corners. As used in this document, “substantially hollow” means at least about 33% of the interior volume of the interbody spinal implant 1 is vacant. The implant 1 includes at least one vertical aperture 60 that extends the entire height of the implant body. As illustrated in the top view of FIG. 2, the vertical aperture 60 further defines a transverse rim 100 having a greater posterior portion thickness 55 than an anterior portion thickness 45.

In at least one embodiment, the opposing lateral sides 30 and the anterior portion 40 have a rim thickness of about 5 mm, while the posterior portion 50 has a rim thickness of about 7 mm. Thus, the rim posterior portion thickness 55 may allow for better stress sharing between the implant 1 and the adjacent vertebral endplates and helps to compensate for the weaker posterior endplate bone. In certain embodiments, the transverse rim 100 has a generally large surface area and contacts the vertebral endplate. The transverse rim 100 may act to better distribute contact stresses upon the implant 1, and hence minimize the risk of subsidence while maximizing contact with the apophyseal supportive bone. It is also possible for the transverse rim 100 to have a substantially constant thickness (i.e., for the anterior portion thickness 45 to be substantially the same as the posterior portion thickness 55) or, in fact, for the posterior portion 50 to have a rim thickness less than that of the opposing lateral sides 30 and the anterior portion 40. Some studies have challenged the characterization of the posterior endplate bone as weaker.

It is generally believed that the surface of an implant determines its ultimate ability to integrate into the surrounding living bone. Without being limited by theory, it is hypothesized that the cumulative effects of at least implant composition, implant surface energy, and implant surface roughness play a major role in the biological response to, and osteointegration of, an implant device. Thus, implant fixation may depend, at least in part, on the attachment and proliferation of osteoblasts and like-functioning cells upon the implant surface. Still further, it appears that these cells attach more readily to relatively rough surfaces rather than smooth surfaces. In this manner, a surface may be bioactive due to its ability to facilitate cellular attachment and osteointegration. The surface roughened topography 80 may better promote the osteointegration of certain embodiments of the present invention. The surface roughened topography 80 may also better grip the vertebral endplate surfaces and inhibit implant migration upon placement and seating.

Accordingly, the implant 1 further includes the roughened topography 80 on at least a portion of its top and bottom surfaces 10, 20 for gripping adjacent bone and inhibiting migration of the implant 1. The roughened topography 80 may be obtained through a variety of techniques including, without limitation, chemical etching, shot peening, plasma etching, laser etching, or abrasive blasting (such as sand or grit blasting). In at least one embodiment, the interbody spinal implant 1 may be comprised of titanium, or a titanium alloy, having the surface roughened topography 80. The surfaces of the implant 1 are preferably bioactive.

In a preferred embodiment of the present invention, the roughened topography 80 is obtained via the repetitive masking and chemical and electrochemical milling processes described in U.S. Pat. No. 5,258,098; U.S. Pat. No. 5,507,815; U.S. Pat. No. 5,922,029; and U.S. Pat. No. 6,193,762. Each of these patents is incorporated in this document by reference. Where the invention employs chemical etching, the surface is prepared through an etching process which utilizes the random application of a maskant and subsequent etching of the metallic substrate in areas unprotected by the maskant. This etching process is repeated a number of times as necessitated by the amount and nature of the irregularities required for any particular application. Control of the strength of the etchant material, the temperature at which the etching process takes place, and the time allotted for the etching process allow fine control over the resulting surface produced by the process. The number of repetitions of the etching process can also be used to control the surface features.

By way of example, an etchant mixture of nitric acid (HNO₃) and hydrofluoric (HF) acid may be repeatedly applied to a titanium surface to produce an average etch depth of about 0.53 mm. Interbody spinal implants, in accordance with preferred embodiments of the present invention, may be comprised of titanium, or a titanium alloy, having an average surface roughness of about 100 μm. Surface roughness may be measured using a laser profilometer or other standard instrumentation.

In another example, chemical modification of the titanium implant surfaces can be achieved using HF and a combination of hydrochloric acid and sulfuric acid (HCl/H₂SO₄). In a dual acid etching process, the first exposure is to HF and the second is to HCl/H₂SO₄. Chemical acid etching alone of the titanium implant surface has the potential to greatly enhance osseointegration without adding particulate matter (e.g., hydroxyapatite) or embedding surface contaminants (e.g., grit particles).

Certain embodiments of the implant 1 are generally shaped to reduce the risk of subsidence, and improve stability, by maximizing contact with the apophyseal rim of the vertebral endplates. Embodiments may be provided in a variety of anatomical footprints having a medial-lateral width ranging from about 32 mm to about 44 mm. Interbody spinal implants, as now taught, generally do not require extensive supplemental or obstructive implant instrumentation to maintain the prepared disc space during implantation. Thus, the interbody spinal implant 1 and associated implantation methods, according to presently preferred aspects of the present invention, allow for larger sized implants as compared with the size-limited interbody spinal implants known in the art. This advantage allows for greater medial-lateral width and correspondingly greater contact with the apophyseal rim.

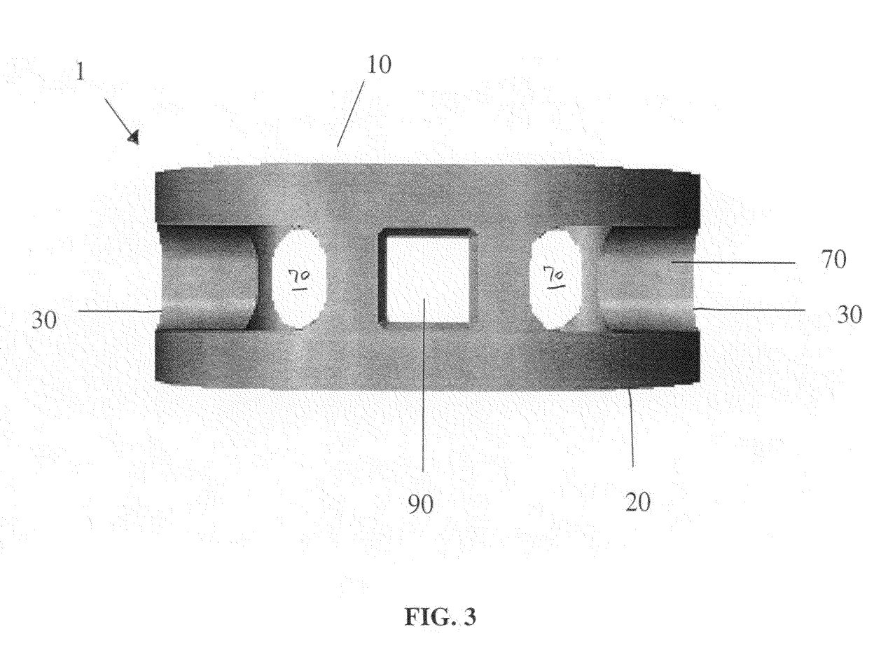

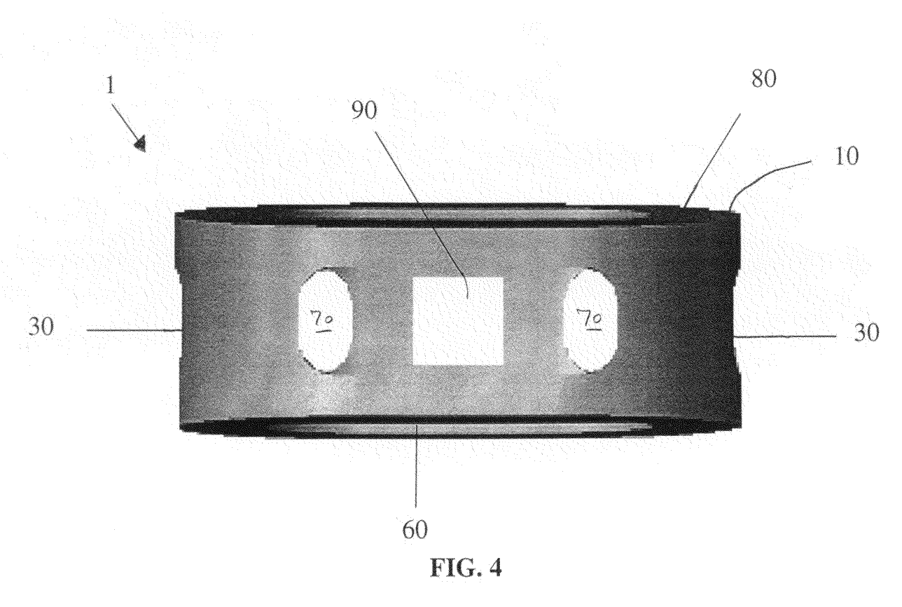

FIG. 3 depicts an anterior view, and FIG. 4 depicts a posterior view, of an embodiment of the interbody spinal implant 1. As illustrated in FIGS. 1 and 3, the implant 1 has an opening 90 in the anterior portion 40. As illustrated in FIGS. 3 and 4, in one embodiment the posterior portion 50 has a similarly shaped opening 90. In another embodiment, as illustrated in FIG. 1, only the anterior portion 40 has the opening 90 while the posterior portion 50 has an alternative opening 92 (which may have a size and shape different from the opening 90).

The opening 90 has a number of functions. One function is to facilitate manipulation of the implant 1 by the caretaker. Thus, the caretaker may insert a surgical tool into the opening 90 and, through the engagement between the surgical tool and the opening 90, manipulate the implant 1. The opening 90 may be threaded to enhance the engagement.

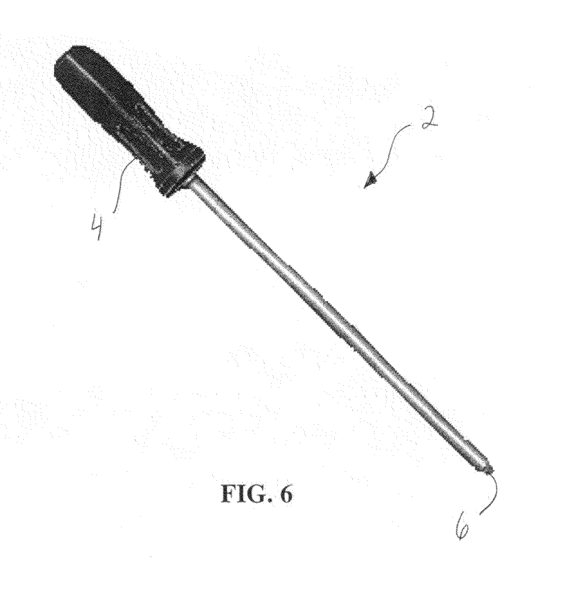

FIG. 6 shows an exemplary surgical tool, specifically an implant holder 2, to be used with certain embodiments of the interbody spinal implant 1. Typically, the implant holder 2 has a handle 4 that the caretaker can easily grasp and an end 6 that engages the opening 90. The end 6 may be threaded to engage corresponding threads in the opening 90. The size and shape of the opening 90 can be varied to accommodate a variety of tools. Thus, although the opening 90 is substantially square as illustrated in FIGS. 1, 3, and 4, other sizes and shapes are feasible.

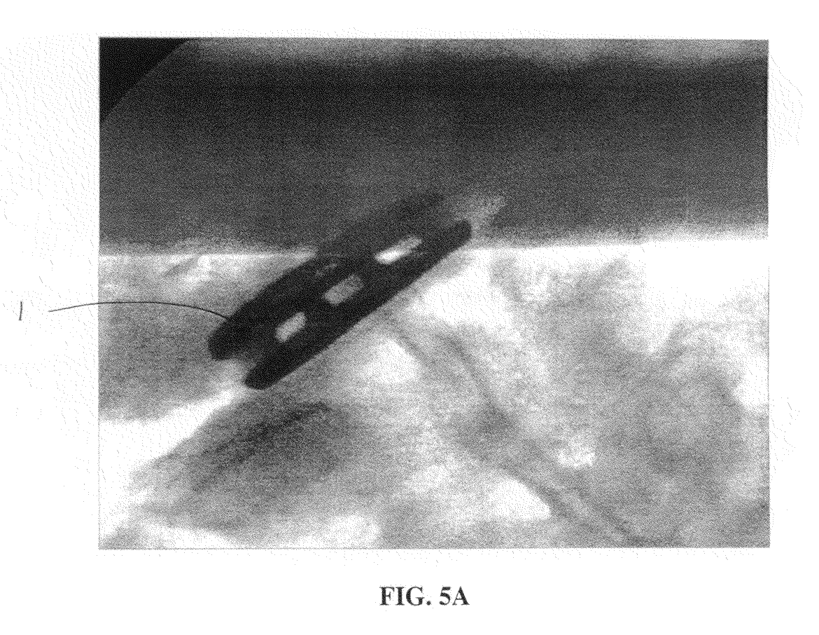

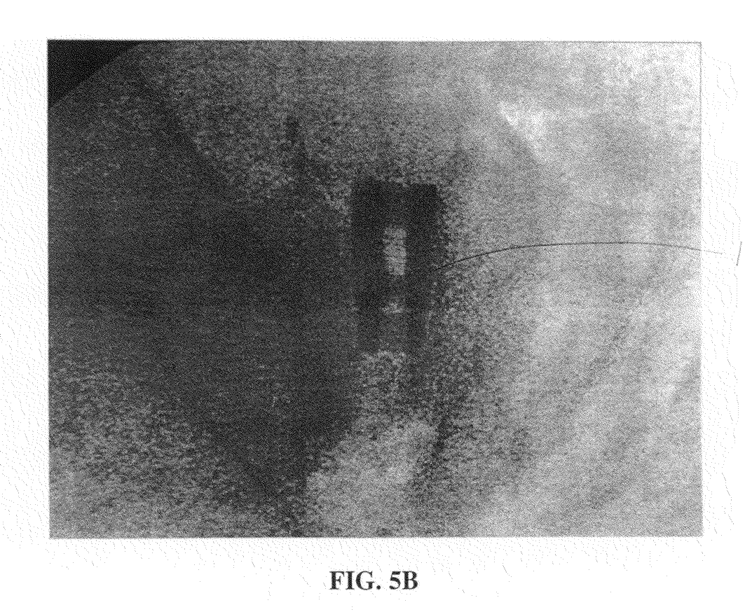

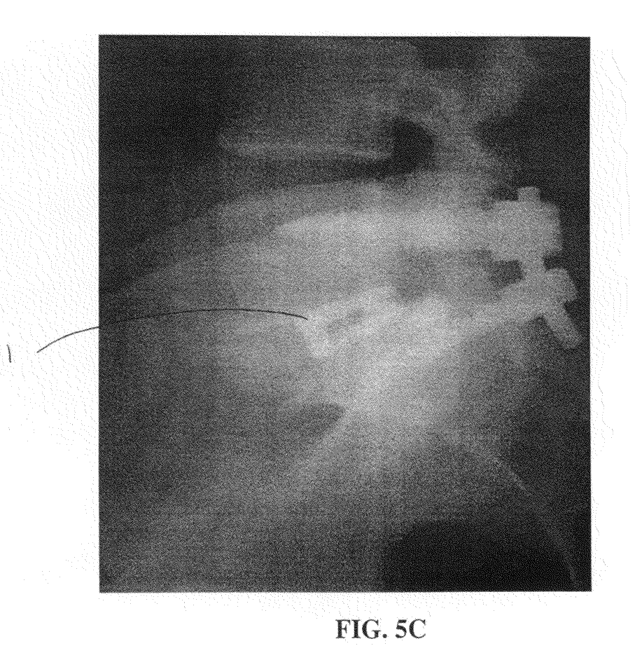

The implant 1 may further include at least one transverse aperture 70 that extends the entire transverse length of the implant body. As shown in FIGS. 5A-5C, these transverse apertures 70 may provide improved visibility of the implant 1 during surgical procedures to ensure proper implant placement and seating, and may also improve post-operative assessment of implant fusion. Still further, the substantially hollow area defined by the implant 1 may be filled with cancellous autograft bone, allograft bone, DBM, porous synthetic bone graft substitute, BMP, or combinations of these materials (collectively, bone graft materials), to facilitate the formation of a solid fusion column within the spine of a patient.

The anterior portion 40, or trailing edge, of the implant 1 is preferably generally greater in height than the opposing posterior portion 50. Accordingly, the implant 1 may have a lordotic angle to facilitate sagittal alignment. The implant 1 may better compensate, therefore, for the generally less supportive bone found in the posterior regions of the vertebral endplate. The posterior portion 50 of the interbody implant 1, preferably including the posterior-lateral corners, may also be highly radiused, thus allowing for ease of implantation into the disc space. Thus, the posterior portion 50 may have a generally blunt nosed profile. The anterior portion 40 of the implant 1 may also preferably be configured to engage a delivery device, driver, or other surgical tool (and, therefore, may have an opening 90).

As illustrated in FIG. 1, the anterior portion 40 of the implant 1 is substantially flat. Thus, the anterior portion 40 provides a face that can receive impact from a tool, such as a surgical hammer, to force the implant 1 into position. The implant 1 has a sharp edge 8 where the anterior portion 40 meets the top surface 10, where the anterior portion 40 meets the bottom surface 20, or in both locations. The sharp edge or edges 8 function to resist pullout of the implant 1 once it is inserted into position.

Certain embodiments of the present invention are particularly suited for use during interbody spinal implant procedures (or vertebral body replacement procedures) and may act as a final distractor during implantation, thus minimizing the instrument load upon the surgeon. For example, in such a surgical procedure, the spine may first be exposed via an anterior approach and the center of the disc space identified. The disc space is then initially prepared for implant insertion by removing vertebral cartilage. Soft tissue and residual cartilage may then also be removed from the vertebral endplates.

Vertebral distraction may be performed using trials of various-sized embodiments of the interbody spinal implant 1. The determinatively sized interbody implant 1 may then be inserted in the prepared disc space for final placement. The distraction procedure and final insertion may also be performed under fluoroscopic guidance. The substantially hollow area within the implant body may optionally be filled, at least partially, with bone fusion-enabling materials such as, without limitation, cancellous autograft bone, allograft bone, DBM, porous synthetic bone graft substitute, BMP, or combinations of those materials. Such bone fusion-enabling material may be delivered to the interior of the interbody spinal implant 1 using a delivery device mated with the opening 90 in the anterior portion 40 of the implant 1. Interbody spinal implants 1, as now taught, are generally larger than those currently known in the art, and therefore have a correspondingly larger hollow area which may deliver larger volumes of fusion-enabling bone graft material. The bone graft material may be delivered such that it fills the full volume, or less than the full volume, of the implant interior and surrounding disc space appropriately.

In another embodiment of the present invention, an interbody spinal implant 101 is a composite device that combines the benefits of two, separate components: a frame, skeleton, or cage 110 and a body 150. The composite structure of implant 101 advantageously permits the engineering designer of the implant 101 to balance the mechanical characteristics of the overall implant 101. Thus, the implant 101 can achieve the best balance, for example, of strength, resistance to subsidence, and stress transfer to bone graft. Moreover, although it is a relatively wide device designed to engage the ends of the vertebrae, the implant 101 can be inserted with minimal surgical modification. This combination of size and minimal surgical modification is advantageous.

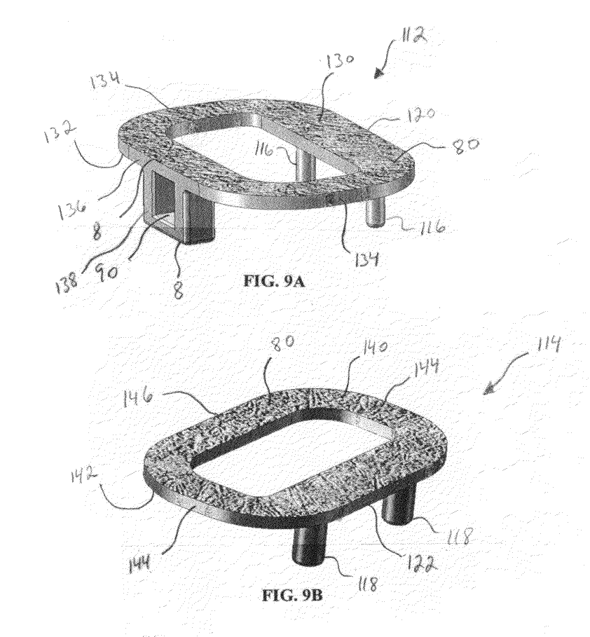

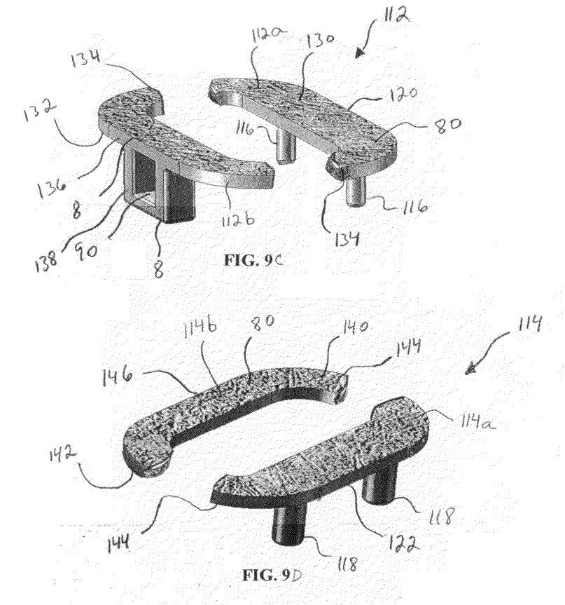

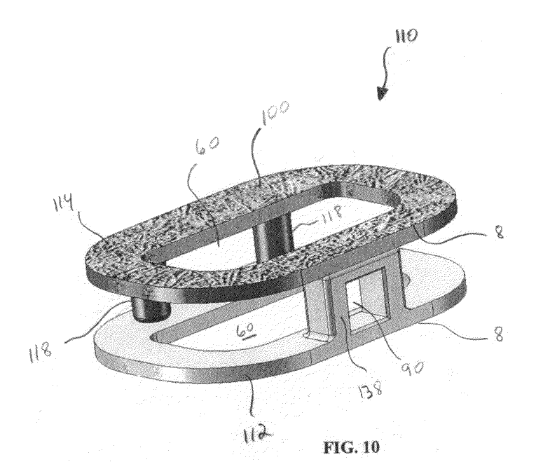

FIGS. 9A and 9B illustrate one embodiment of the cage 110. The cage 110 includes two plates, a top plate 112 (shown in FIG. 9A) and a bottom plate 114 (shown in FIG. 9B). In combination, the top plate 112 and bottom plate 114 form the cage 110. The top plate 112 has a plurality (two or more) of male posts 116 while the bottom plate 114 has a corresponding number of female columns 118. Although two posts 116 and columns 118 are illustrated in FIGS. 9A and 9B, more posts 116 and columns 118 could be provided. In addition, the columns 118 might be provided on the top plate 112 while the posts 116 might be provided on the bottom plate 114. In either case, the posts 116 and columns 118 are designed so that the male posts 116 enter the female columns 118 when the top plate 112 of the cage 110 is assembled with the bottom plate 114 of the cage 110, as shown in FIG. 10. The posts 116 and columns 118 are positioned (typically, although not necessarily) on the posterior portions 120, 122, respectively, of the top plate 112 and bottom plate 114.

The top plate 112 has a top surface 130, a bottom surface 132 which faces the bottom plate 114, opposing lateral sides 134, and opposing anterior 136 and posterior 120 portions. The top surface 130 has a roughened topography 80. The anterior 136 of the top plate 112 includes a substantially flat front face 138, which can absorb impact sufficient to position the implant 101, defining the opening 90 and a sharp edge 8 (as for the previous embodiment illustrated in FIG. 1). In contrast to the substantially flat front face 138, the lateral sides 134 and the posterior 120 of the top plate 112 are rounded to ease placement of the implant 101.

The bottom plate 114 has a bottom surface 140, a top surface 142 which faces the top plate 112, opposing lateral sides 144, and opposing anterior 146 and posterior 122 portions. The bottom surface 140 has a roughened topography 80. The anterior 146 of the bottom plate 114 includes a substantially flat front face corresponding to the front face 138 of the top plate 112 and a sharp edge 8 (shown in FIG. 10). In contrast to the substantially flat front face of the anterior 146, the lateral sides 144 and the posterior 122 of the bottom plate 114 are rounded to ease placement of the implant 101.

FIGS. 9C and 9D illustrate another embodiment of the cage 110. Each plate 112, 114 of the cage 110 in this embodiment includes two, separate sections. FIG. 9C illustrates the top plate 112 of the cage 110 formed as two, separate sections 112a and 112b. Similarly, FIG. 9D illustrates the bottom plate 114 of the cage 110 formed as two, separate sections 114a and 114b. The remaining structure of the implant 101 is provided by the body 150. Thus, less of the material used to create the cage 110 and more of the material used to create the body 150 are incorporated into the implant 101 in the embodiment of FIGS. 9C and 9D. Otherwise, the features of the top plate 112 (shown in FIG. 9A) and the bottom plate 114 (shown in FIG. 9B) are the same for the embodiment of FIGS. 9C and 9D. The structure illustrated in FIGS. 9C and 9D as a cage 110 having four, separate components 112a, 112b, 114a, and 114b gives the designer great flexibility. For example, the designer can minimize such problems as implant subsidence, stress shielding, implant incorporation with vertebral bone, radiographic visualization, and manufacturing cost.

FIG. 10 shows the top plate 112 and the bottom plate 114 in their assembled position to form the cage 110 of the implant 101. As assembled, the cage 110 includes at least one vertical aperture 60 that extends the entire height of the implant 101. The vertical aperture 60 is provided to receive bone graft material and, further, defines a transverse rim 100. The sharp edge or edges 8 function to resist pullout of the implant 101 once it is inserted into position.

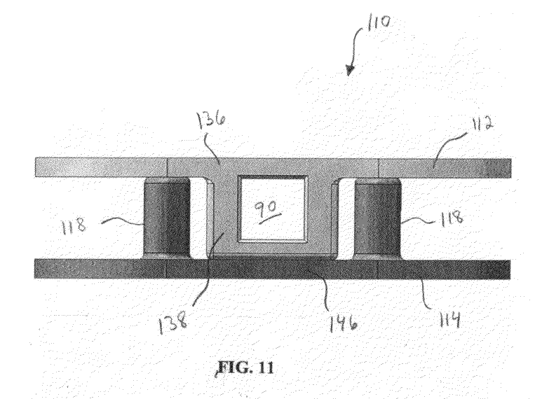

FIG. 11 depicts an anterior view of the assembled cage 110 shown in FIG. 10. As illustrated in FIG. 11, the top plate 112 of the cage 110 is fully seated on the bottom plate 114 of the cage 110. In this position, the male posts 116 of the top plate 112 reside fully within the female columns 118 of the bottom plate 114.

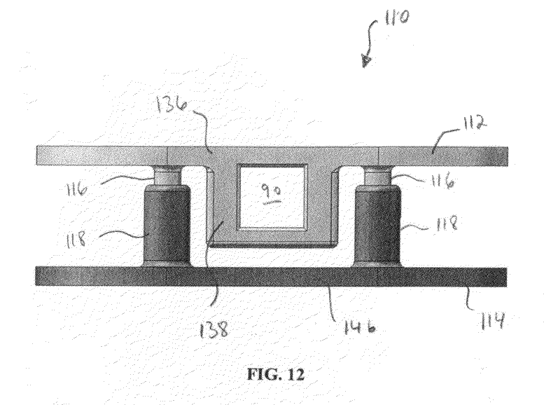

FIG. 12 depicts another anterior view of the assembled cage 110 shown in FIG. 10, illustrating the telescopic feature of the present invention. As illustrated in FIG. 12, the top plate 112 of the cage 110 is slightly raised with respect to the bottom plate 114 of the cage 110. In this position, the male posts 116 of the top plate 112 extend partially outside the female columns 118 of the bottom plate 114.

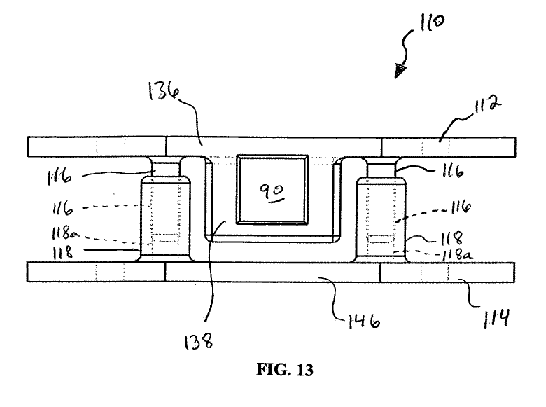

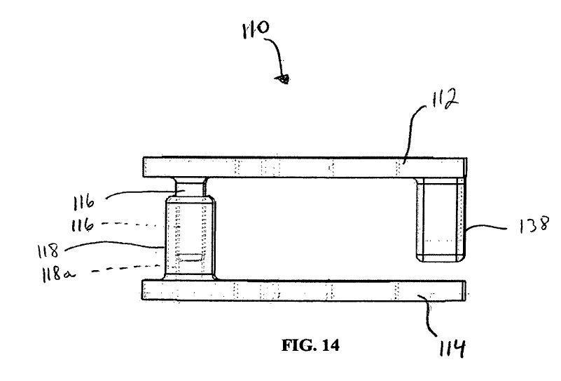

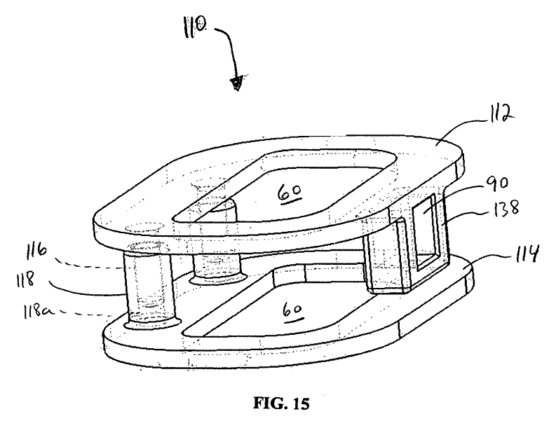

FIG. 13 is the same anterior view of the assembled cage 110 shown in FIG. 12, but depicts the interior channels 118a that extend vertically within each of the female columns 118. The channels 118a receive the male posts 116 of the top plate 112. FIG. 14 is a lateral side view, and FIG. 15 is a perspective view, of the assembled cage 110 shown in FIG. 13.

The telescoping design of the implant 101 according to the present invention allows the implant 101 to change in size while in position within the patient. Thus, implant 101 permits micromotion, namely small but decipherable amounts of rotation and translation, to facilitate the process of patient healing and enhance stability. Vertebral bodies can vibrate and deflect; so, too, can the implant 101. Conventional devices do not permit such micromotion. It is also possible, of course, to take advantage of the telescoping design of the implant 101 outside the context of a dynamic implant: the implant 101 could be adjusted to a final position, and fixed in that position, before implantation.

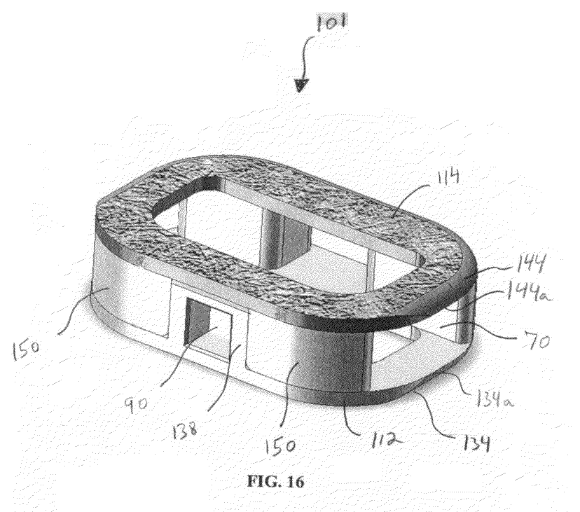

FIG. 16 shows the implant 101 after the cage 110, including the top plate 112 and the bottom plate 114 in their assembled position, is combined with the body 150. As illustrated, the implant 101 further includes at least one transverse aperture 70 that extends the entire transverse length of the implant 101. The transverse aperture 70 may provide improved visibility of the implant 101 during surgical procedures to ensure proper implant placement and seating, and may also improve post-operative assessment of implant fusion. More specifically, the transverse aperture 70 provides a large radiographic window.

As illustrated in FIG. 16, the lateral side 134 of the top plate 112 of the cage 110 may include a rounded edge 134a. Similarly, the lateral side 144 of the bottom plate 114 of the cage 110 may include a rounded edge 144a. The rounded edges 134a, 144a facilitate placement of the implant 101.

The top plate 112 and bottom plate 114 of the cage 110 are typically made of metal, a metal alloy, or both. Titanium and its alloys are generally preferred. Most preferred is Grade 5 titanium, which is the workhorse of all the titanium grades. It is also known as Ti-6AL-4V or simply Ti 6-4. Its high strength, light weight, and corrosion resistance enables Ti 6-4 to be used in many applications. Such materials give the implant 101 suitable strength, biocompatibility, and structural integrity and may better resist fracture during implantation by impact.

The body 150 of the implant 101 is typically made of a polymer or a ceramic material. PEEK is generally preferred. Such materials give implant 101 suitable stiffness. The PEEK material has a modulus of elasticity somewhat less than that of titanium and, therefore, matches the stiffness of bone better than titanium. Moreover, PEEK is radiolucent, facilitating the process of securing information via X-ray, and is close to actual bone in strength.

The composite spinal implant 101 offers a number of advantages. Specifically, for example, the composite design of the implant 101 renders it relatively easy to make implants of different sizes. The same metal top and bottom plates 112, 114 can be combined with bodies 150 of different heights. Thus, a reduction in the per-piece price of the implant 101 can be realized.

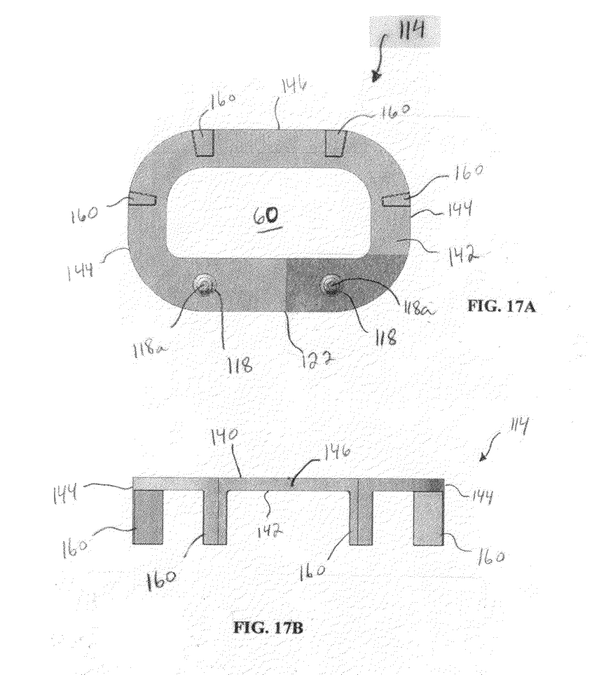

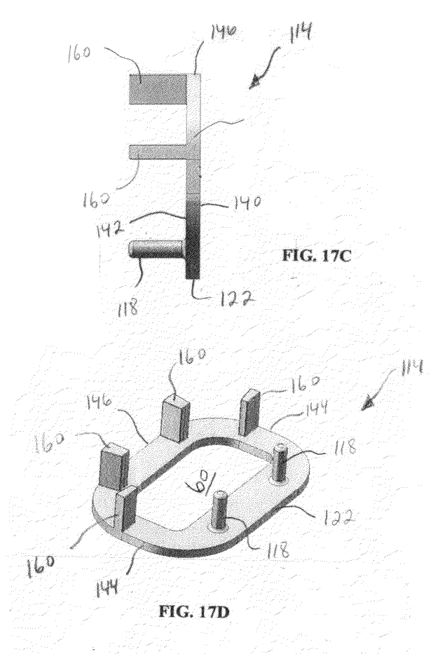

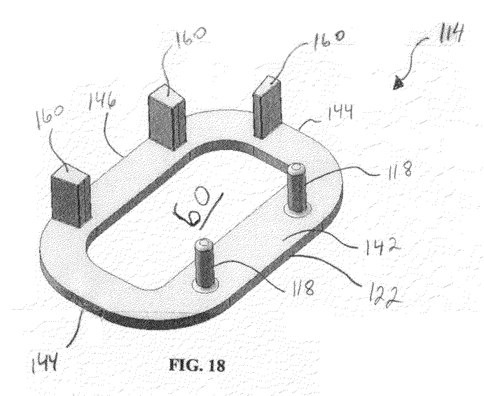

FIGS. 17A, 17B, 17C, and 17D illustrate yet another embodiment of the present invention. In this embodiment, the bottom plate 114 of the implant 101 is provided with one or more struts 160. These figures illustrate four struts 160; two struts 160 are located proximate the anterior portion 146 of the bottom plate 114 and two struts 160 are located proximate opposite lateral sides 144 of the bottom plate 114. Any number of struts 160 may be suitable, however, depending upon a particular application. Three struts 160 are illustrated in FIG. 18 (one of the struts 160 located on a lateral side 144 has been eliminated for purposes of example only). Regardless of their number, the struts 160 enhance the structural integrity of the implant 101. Like the posts 116 and columns 118, the struts 160 provide shear resistance. Another function of the struts 160 is to facilitate one or more of anterior, antero-lateral, and lateral implant—depending on the number and location of the struts. Each strut 160 provides a face that can accept force from a tool (e.g., a hammer) during insertion of the implant 101.

Preferably, like the columns 118, the struts 160 are an integral (i.e., formed as one piece or monolithic) part of the bottom plate 114. The height of the struts 160 should be approximately the same as the height of the columns 118. Otherwise, the dimensions (i.e., width and thickness) are subject to design modification depending upon the application. Wedge-shaped struts 160, as illustrated in FIGS. 17A, 17D, and 18, are suitable as one example. Of course, structure similar to struts 160 could be incorporated on the top plate 112 either instead of or in addition to struts 160 on the bottom plate 114.

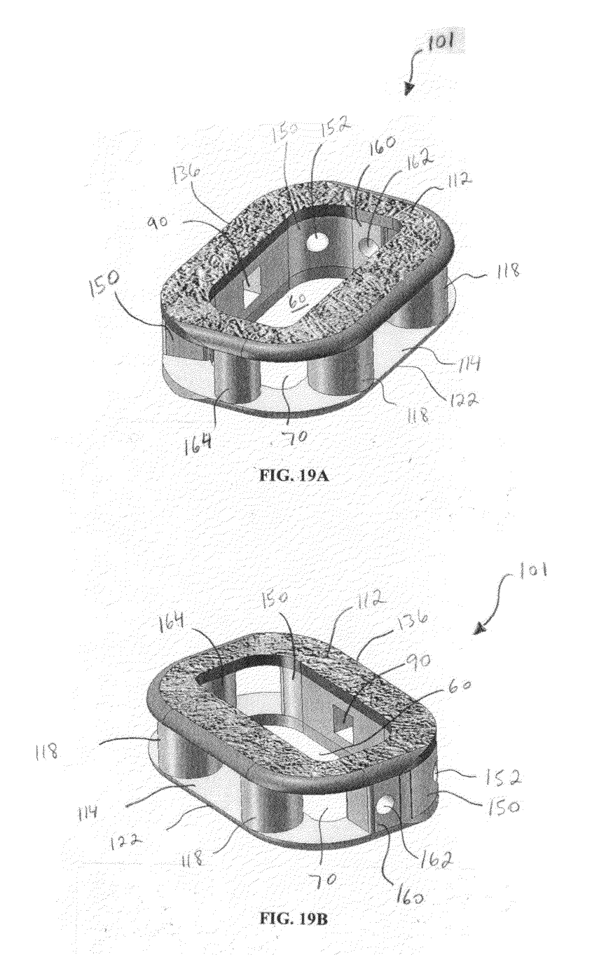

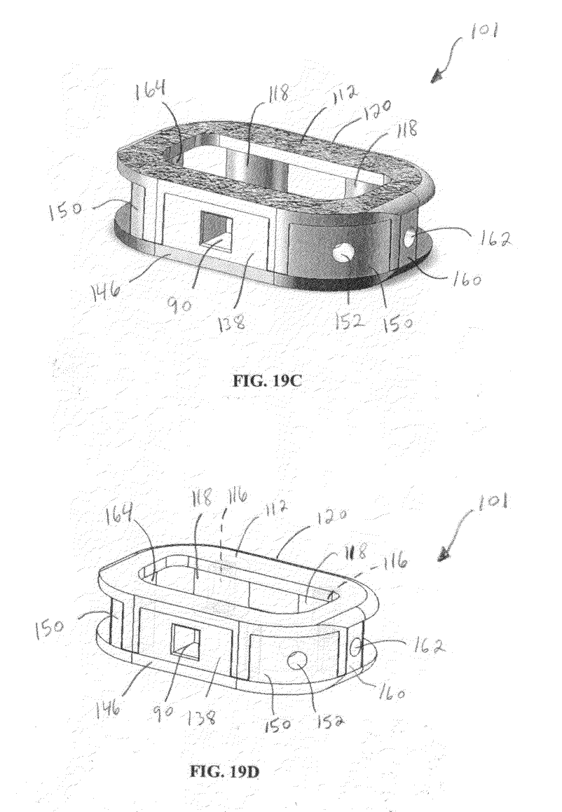

FIGS. 19A, 19B, 19C, and 19D illustrate yet another embodiment of the present invention. In this embodiment, a cylindrical-shaped strut 164 is shown in addition to a wedge-shaped strut 160 as previously illustrated. Further, the wedge-shaped strut 160 is provided with a hole 162 and the body 150 is provided with a hole 152. In alternative embodiments, one or the other of the holes 152, 162 might be eliminated. Like the opening 90, when provided holes 152, 162 have a number of functions. One function is to facilitate manipulation of the implant 101 by the caretaker. Thus, the caretaker may insert a surgical tool into one or both of the holes 152, 162 and, through the engagement between the surgical tool and the holes 152, 162, manipulate the implant 101. One or both of the holes 152, 162 may be threaded to enhance the engagement. The holes 152, 162 facilitate antero-lateral and lateral implant of the spinal implant 101.

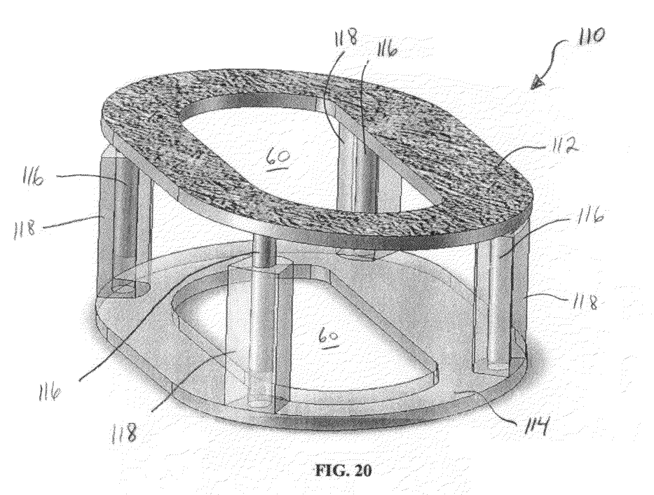

FIG. 20 is a perspective view of still another embodiment of the present invention. The body 150 has been omitted from FIG. 20, although the body 150 would be added to the cage 110 before application, so that the features of the cage 110 can be more clearly seen. In this embodiment, the body 150 would likely (although not necessarily) be provided with a hole 152 because the front face 138 of the top plate 112, and the opening 90 of the front face 138, are not included in the cage 110. Thus, the hole 152 would be used to manipulate the implant 101. The absence of the front face 138 opens up the cage 110 even more than some of the earlier embodiments. Therefore, this embodiment can incorporate more PEEK material, more graft material, or more of both types of material. This embodiment also may improve the visibility of the implant 101 to such detection techniques as X-rays, for example.

The embodiment illustrated in FIG. 20 has four telescoping posts 116 on the top plate 112 and four corresponding columns 118 on the bottom plate 114. The columns 118 are shaped (e.g., as wedges) to accommodate the impact of a tool or instrument during placement of the implant 101. Of course, the number of posts 116 and columns 118 can be varied depending upon a particular application.

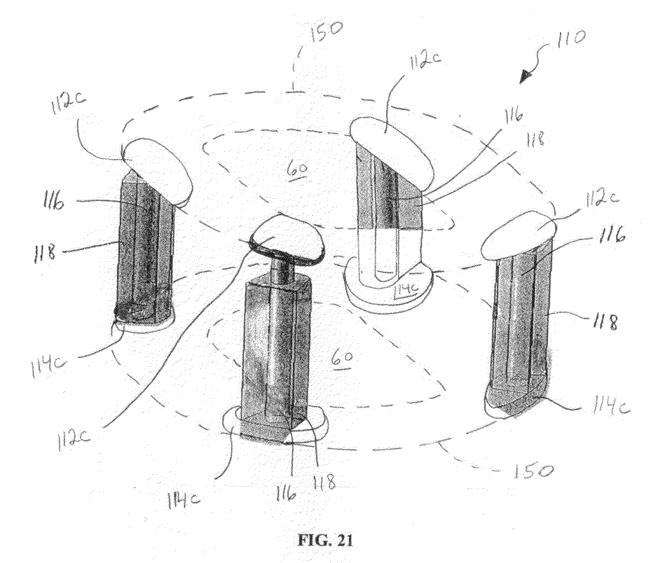

FIG. 21 is a perspective view of the cage 110 forming a further embodiment of the interbody spinal implant 101 according to the present invention. FIG. 21 depicts a cage 110 having four posts 116 on abbreviated top plate sections 112c and four corresponding columns 118 on abbreviated bottom plate sections 114c. As shown, the cage 110 illustrated in FIG. 21 eliminates much of the top plate 112 and the bottom plate 114 of previous embodiments. Preferably, the posts 116 are integral with the top plate sections 112c and the columns 118 are integral with the bottom plate sections 114c. The body 150 has been omitted from FIG. 21, although the periphery of the body 150 is shown in dashed lines, so that the features of the cage 110 can be more clearly seen. The body 150 would be added to the cage 110 before application.

In the embodiment illustrated in FIG. 21, less of the material used to create the cage 110 and more of the material used to create the body 150 are incorporated into the implant 101. The structure illustrated in FIG. 21 opens up the cage 110 even more open than some of the earlier embodiments and gives the designer great flexibility. For example, this embodiment can incorporate more PEEK or Hedrocel material, more graft material, or more of both types of material. This flexibility allows the designer to minimize such problems as implant subsidence, stress shielding, implant incorporation with vertebral bone, radiographic visualization, and manufacturing cost.

The embodiment illustrated in FIG. 21 has four telescoping posts 116 on the top plate sections 112c and four corresponding columns 118 on the bottom plate sections 114c. The columns 118 are shaped (e.g., as wedges) to accommodate the impact of a tool or instrument during placement of the implant 101. Of course, the number of posts 116 and columns 118 can be varied depending upon a particular application.

Certain embodiments of the implant 101 are generally shaped (i.e., made wide) to maximize contact with the apophyseal rim of the vertebral endplates. They are designed to be impacted between the endplates, with fixation to the endplates created by an interference fit and annular tension. Thus, the implant 101 is shaped and sized to spare the vertebral endplates and leave intact the hoop stress of the endplates. A wide range of sizes are possible to capture the apophyseal rim, along with a broad width of the peripheral rim, especially in the posterior region. It is expected that such designs will lead to reduced subsidence. Seven degrees of lordosis are built into the implant 101 to help restore sagittal balance.

When the ring-shaped, endplate-sparing, spinal implant 101 seats in the disc space against the apophyseal rim, it should still allow for deflection of the endplates like a diaphragm. This means that, regardless of the stiffness of the spinal implant 101, the bone graft material inside the spinal implant 101 receives load due to the micro-motion of the endplates, leading to healthy fusion. The vertical load in the human spine is transferred though the peripheral cortex of the vertebral bodies. By implanting an apophyseal-supporting inter-body implant 101, the natural biomechanics may be better preserved than for conventional devices. If this is true, the adjacent vertebral bodies should be better preserved by the implant 101, hence reducing the risk of adjacent segment issues.

In addition, the dual-acid etched roughened topography 80 of the top surface 130 and the bottom surface 140, along with the broad surface area of contact with the end-plates, is expected to yield a high anterior-posterior pull-out force in comparison to conventional designs. As enhanced by the sharp edges 8, a pull-out strength of up to 3,000 nt may be expected. The roughened topography 80 creates a biological bond with the end-plates over time, which should enhance the quality of fusion to the bone. Also, the in-growth starts to happen much earlier than the bony fusion. The center of the implant 101 remains open to receive bone graft material and enhance fusion. Therefore, it is possible that patients might be able to achieve a full activity level sooner than for conventional designs.

The spinal implant 101 according to the present invention offers several advantages relative to conventional devices. Such conventional devices include, among others, ring-shaped cages made of allograft bone material, threaded titanium cages, and ring-shaped cages made of PEEK or carbon fiber. Several of the advantages are summarized with respect to each conventional device, in turn, as follows.

1. Advantages Over Allograft Bone Material Cages

The spinal implant 101 is easier to use than ring-shaped cages made of allograft bone material. For example, it is easier to prepare the graft bed, relative to the allograft cage, for the spinal implant 101. And ring allograft cages typically are not sufficiently wide to be implanted on the apophasis. The spinal implant 101 offers a large internal area for bone graft material and does not require graft preparation, cutting, or trimming. The central aperture 60 of the spinal implant 101 can be filled with cancellous allograft, porous synthetic bone graft substitute (such as the material offered by Orthovita, Inc., Malvern, Pa., under the Vitoss trademark), or BMP. The process of healing the bone can proceed by intra-membranous ossification rather than the much slower process of enchondral ossification.

The spinal implant 101 is generally stronger than allograft cages. In addition, the risk of osteolysis (or, more generally, disease transmission) is minimal with the spinal implant 101 because titanium is osteocompatible. The titanium of the spinal implant 101 is unaffected by BMP; there have been reports that BMP causes resorption of allograft bone.

2. Advantages Over Threaded Titanium Cages

In contrast to conventional treaded titanium cages, which offer little bone-to-bone contact (about 9%), the spinal implant 101 has a much higher bone-to-bone contact area and commensurately little metal-to-bone interface. Unlike threaded titanium cages which have too large a diameter, the spinal implant 101 can be relatively easily used in “tall” disc spaces. The spinal implant 101 can also be used in either a “stand alone” manner at L5-S1 in collapsed discs or as an adjunct to a 360-degree fusion providing anterior column support.

The spinal implant 101 offers safety advantages over conventional threaded titanium cages. The spinal implant 101 is also easier to implant, avoiding the tubes necessary to insert some conventional cages, and easier to center. Without having to put a tube into the disc space, the vein can be visualized by both the spine surgeon and the vascular surgeon while working with the spinal implant 101. Anterior-posterior (AP) fluoroscopy can easily be achieved with trial before implanting the spinal implant 101, ensuring proper placement. The smooth lateral sides and posterior of the spinal implant 101 facilitate insertion and enhance safety. No reaming of the endplate, which weakens the interface between the endplate and the cage, is necessary for the spinal implant 101. Therefore, no reamers or taps are generally needed to insert and position the spinal implant 101.

3. Advantages Over PEEK/Carbon Fiber Cages

Cages made of PEEK or carbon fiber cannot withstand the high impact forces needed for implantation, especially in a collapsed disc or spondylolisthesis situation, without secondary instruments. In contrast, the spinal implant 101 avoids the need for secondary instruments. Moreover, relative to PEEK or carbon fiber cages, the spinal implant 101 provides better distraction through endplate sparing and being designed to be implanted on the apophysis (the bony protuberance of the human spine). The titanium of the top plate 112 and the bottom plate 114 of the spinal implant 101 binds to bone with a mechanical (knawling) and a chemical (a hydrophilic) bond. In contrast, bone repels PEEK and such incompatibility can lead to locked pesudoarthrosis.

### Example Surgical Methods

The following examples of surgical methods are included to more clearly demonstrate the overall nature of the invention. These examples are exemplary, not restrictive, of the invention.

Certain embodiments of the present invention are particularly suited for use during interbody spinal implant procedures currently known in the art. For example, the disc space may be accessed using a standard mini open retroperitoneal laparotomy approach. The center of the disc space is located by AP fluoroscopy taking care to make sure the pedicles are equidistant from the spinous process. The disc space is then incised by making a window in the annulus for insertion of certain embodiments of the spinal implant 1, 101 (a 32 or 36 mm window in the annulus is typically suitable for insertion). The process according to the present invention minimizes, if it does not eliminate, the cutting of bone. The endplates are cleaned of all cartilage with a curette, however, and a size-specific rasp (or broach) may then be used.

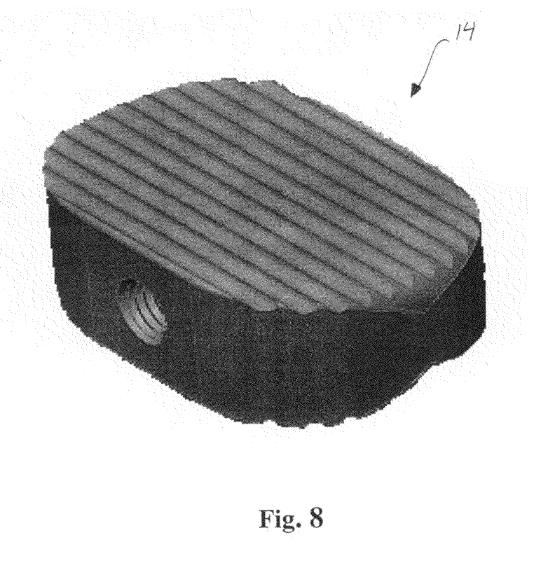

FIG. 8 shows an exemplary rasp 14 used during certain methods of implantation. Typically, either a 32 mm or a 36 mm rasp 14 is used. A single rasp 14 is used to remove a minimal amount of bone. A lateral c-arm fluoroscopy can be used to follow insertion of the rasp 14 in the posterior disc space. The smallest height rasp 14 that touches both endplates (e.g., the superior and inferior endplates) is first chosen. After the disc space is cleared of all soft tissue and cartilage, distraction is then accomplished by using distractors (also called implant trials or distraction plugs). It is usually possible to distract 2-3 mm higher than the rasp 14 that is used because the disk space is elastic.

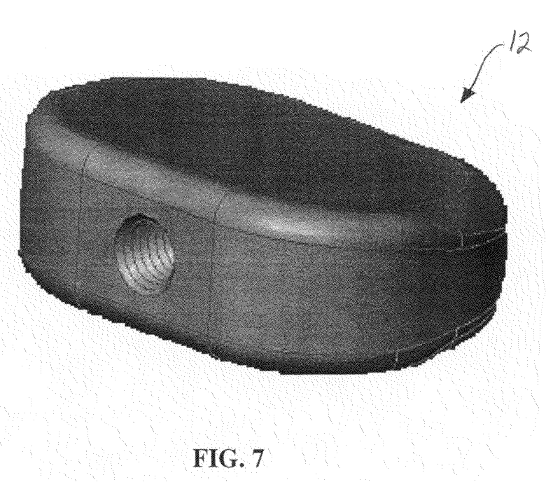

FIG. 7 shows an exemplary distractor 12 used during certain methods of implantation. The implant trials, or distractors 12, are solid polished blocks which have a peripheral geometry identical to that of the implant 1, 101. These distractor blocks may be made in various heights to match the height of the implant 1, 101. The disc space is adequately distracted by sequentially expanding it with distractors 12 of progressively increasing heights. The distractor 12 is then left in the disc space and the centering location may be checked by placing the c-arm back into the AP position. If the location is confirmed as correct (e.g., centered), the c-arm is turned back into the lateral position. The spinal implant 1, 101 is filled with autologous bone graft or bone graft substitute. The distractor 12 is removed and the spinal implant 1, 101 is inserted under c-arm fluoroscopy visualization. The process according to the present invention does not use a secondary distractor; rather, distraction of the disc space is provided by the spinal implant 1, 101 itself (i.e., the implant 1, 101 itself is used as a distractor).

Use of a size-specific rasp 14, as shown in FIG. 8, preferably minimizes removal of bone, thus minimizing impact to the natural anatomical arch, or concavity, of the vertebral endplate while preserving much of the apophyseal rim. Preservation of the anatomical concavity is particularly advantageous in maintaining biomechanical integrity of the spine. For example, in a healthy spine, the transfer of compressive loads from the vertebrae to the spinal disc is achieved via hoop stresses acting upon the natural arch of the endplate. The distribution of forces, and resultant hoop stress, along the natural arch allows the relatively thin shell of subchondral bone to transfer large amounts of load.

During traditional fusion procedures, the vertebral endplate natural arch may be significantly removed due to excessive surface preparation for implant placement and seating. This is especially common where the implant is to be seated near the center of the vertebral endplate or the implant is of relatively small medial-lateral width. Breaching the vertebral endplate natural arch disrupts the biomechanical integrity of the vertebral endplate such that shear stress, rather than hoop stress, acts upon the endplate surface. This redistribution of stresses may result in subsidence of the implant into the vertebral body.

Preferred embodiments of the present surgical method minimize endplate bone removal on the whole, while still allowing for some removal along the vertebral endplate far lateral edges where the subchondral bone is thickest. Still further, certain embodiments of the present interbody spinal implant 1, 101 include smooth, rounded, and highly radiused posterior portions and lateral sides which may minimize extraneous bone removal for endplate preparation. Thus, interbody surgical implants 1, 101 and methods of using them, as now taught, are particularly useful in preserving the natural arch of the vertebral endplate and minimizing the chance of implant subsidence.

Because the endplates are spared during the process of inserting the spinal implant 1, 101, hoop stress of the inferior and superior endplates is maintained. Spared endplates allow the transfer of axial stress to the apophasis. Endplate flexion allows the bone graft placed in the interior of the spinal implant 1, 101 to accept and share stress transmitted from the endplates. In addition, spared endplates minimize the concern that BMP might erode the cancellous bone.

Interbody spinal implants 1, 101 of the present invention are durable and can be impacted between the endplates with standard instrumentation. Therefore, certain embodiments of the present invention may be used as the final distractor during implantation. In this manner, the disc space may be under-distracted (e.g., distracted to some height less than the height of the interbody spinal implant 1, 101) to facilitate press-fit implantation. Further, certain embodiments of the current invention having a smooth and rounded posterior portion (and lateral sides) may facilitate easier insertion into the disc space. Still further, those embodiments having a surface roughened topography 80, as now taught, may lessen the risk of excessive bone removal during distraction as compared to implants having teeth, ridges, or threads currently known in the art even in view of a press-fit surgical distraction method. Nonetheless, once implanted, the interbody surgical implants 1, 101, as now taught, may provide secure seating and prove difficult to remove. Thus, certain embodiments of the present interbody spinal implant 1, 101 may maintain a position between the vertebral endplates due, at least in part, to resultant annular tension attributable to press-fit surgical implantation and, post-operatively, improved osteointegration at the top surface 10, 130, the bottom surface 20, 140, or both top and bottom surfaces.

As previously mentioned, surgical implants and methods, as now taught, tension the vertebral annulus via distraction. These embodiments and methods may also restore spinal lordosis, thus improving sagittal and coronal alignment. Implant systems currently known in the art require additional instrumentation, such as distraction plugs, to tension the annulus. These distraction plugs require further tertiary instrumentation, however, to maintain the lordotic correction during actual spinal implant insertion. If tertiary instrumentation is not used, then some amount of lordotic correction may be lost upon distraction plug removal. Interbody spinal implants 1, 101, according to certain embodiments of the present invention, are particularly advantageous in improving spinal lordosis without the need for tertiary instrumentation, thus reducing the instrument load upon the surgeon. This reduced instrument load may further decrease the complexity, and required steps, of the implantation procedure.

Certain embodiments of the spinal implants 1, 101 may also reduce deformities (such as isthmic spondylolythesis) caused by distraction implant methods. Traditional implant systems require secondary or additional instrumentation to maintain the relative position of the vertebrae or distract collapsed disc spaces. In contrast, interbody spinal implants 1, 101, as now taught, may be used as the final distractor and thus maintain the relative position of the vertebrae without the need for secondary instrumentation.

Certain embodiments collectively comprise a family of implants, each having a common design philosophy. These implants and the associated surgical technique have been designed to address the ten, separate challenges associated with the current generation of traditional anterior spinal fusion devices listed above in the Background section of this document. Each of these challenges is addressed in turn and in the order listed above.

1. End-Plate Preparation

Embodiments of the present invention allow end-plate preparation with custom-designed rasps 14. These rasps 14 have a geometry matched with the geometry of the implant. The rasps 14 conveniently remove cartilage from the endplates and remove minimal bone, only in the postero-lateral regions of the vertebral end-plates. It has been reported in the literature that the end-plate is the strongest in postero-lateral regions.

2. Implant Difficulty

After desired annulotomy and discectomy, embodiments of the present invention first adequately distract the disc space by inserting (through impaction) and removing sequentially larger sizes of very smooth distractors, which have size matched with the size of the available implants 1, 101. Once adequate distraction is achieved, the surgeon prepares the end-plate with a size-specific rasp 14. There is no secondary instrumentation required to keep the disc space distracted while the implant 1, 101 is inserted, as the implant 1, 101 has sufficient mechanical strength that it is impacted into the disc space. In fact, the height of the implant 1, 101 is about 1 mm greater than the height of the rasp 14 used for end-plate preparation, to create some additional tension in the annulus by implantation, which creates a stable implant construct in the disc space.

The implant geometry has features which allow it to be implanted via any one of an anterior, antero-lateral, or lateral approach, providing tremendous intra-operative flexibility of options. The implant 1, 101 is designed such that all the impact loads are applied only to the titanium part of the construct. Thus, the implant 1, 101 has adequate strength to allow impact. The sides of the implant 1, 101 have smooth surfaces to allow for easy implantation and, specifically, to prevent “binding” of the implant 1, 101 to soft tissues during implantation.

3. Materials of Construction

The present invention encompasses a number of different implants 1, 101, including a one-piece, titanium-only implant 1 and a composite implant 101 formed of top and bottom plates 112, 114 (components) made out of titanium. The surfaces exposed to the vertebral body are dual acid etched to allow for bony in-growth over time, and to provide resistance against expulsion. The top and bottom titanium plates 112, 114 are assembled together and, while maintaining them apart at a desired distance which is different for implants of different heights, the whole construct is injection molded with PEEK. The net result is a composite implant of desired height. This implant 101 has engineered stiffness for its clinical application. The composite implant 101 is designed so that all impact forces during implantation are borne by the titanium (i.e., metal) components. Also, the titanium construct withstands all physiologic loads in all directions, except for axial loading. The axial load is borne by the PEEK component of the construct.

It is believed that an intact vertebral end-plate deflects like a diaphragm under axial compressive loads generated due to physiologic activities. If a spinal fusion implant is inserted in the prepared disc space via a procedure which does not destroy the end-plates, and if the implant contacts the end-plates only peripherally, the central dome of the end-plates can still deflect under physiologic loads. This deflection of the dome can pressurize the bone graft material packed inside the spinal implant, hence allowing it to heal naturally. The implant 1, 101 designed according to certain embodiments of the present invention allows the vertebral end-plate to deflect and allows healing of the bone graft into fusion.

4. Implant Expulsion

The anterior face of the implant 1, 101 according to certain embodiments of the present invention has sharp edges 8. These edges 8 tend to dig “into” the end-plates slightly and help to resist expulsion. The top and bottom surfaces of the implant are made out of titanium and are dual acid etched. The dual acid etching process creates a highly roughened texture on these surfaces, which generates tremendous resistance to expulsion. The width of these dual acid etched surfaces is very broad and creates a large area of contact with the vertebral end-plates, further increasing the resistance to expulsion.

5. Implant Subsidence

The implant 1, 101 according to certain embodiments of the present invention has a large foot-print, and offers several sizes. Because there is no secondary instrument required to maintain distraction during implantation, all the medial-lateral (ML) exposure is available as implantable ML width of the implant. This feature allows the implant to contact the vertebral end-plates at the peripheral apophyseal rim, where the end-plates are the strongest and least likely to subside.

Further, there are no teeth on the top and bottom surfaces (teeth can create stress risers in the end-plate, encouraging subsidence). Except for the anterior face, all the implant surfaces have heavily rounded edges, creating a low stress contact with the end-plates. The wide rim of the top and bottom surfaces, in contact with the end-plates, creates a low-stress contact due to the large surface area. Finally, the implant construct has an engineered stiffness to minimize the stiffness mismatch with the vertebral body which it contacts.

6. Insufficient Room for Bone Graft

As mentioned, the implant 1, 101 according to certain embodiments of the present invention has a large foot-print. In addition, titanium provides high strength for a small volume. In combination, the large foot-print along with the engineered use of titanium allows for a large volume of bone graft to be placed inside the implant.

7. Stress Shielding

As stated above, it is believed that an intact vertebral end-plate deflects like a diaphragm under axial compressive loads generated due to physiologic activities. If a spinal fusion implant is inserted in the prepared disc space via a procedure which does not destroy the end-plate, and if the implant contacts the end-plates only peripherally, the central dome of the end-plates can still deflect under physiologic loads. This deflection of the dome can pressurize the bone graft material packed inside the spinal implant, hence allowing it to heal naturally. The implant 1, 101 according to certain embodiments of the present invention allows the vertebral end-plate to deflect and facilitates healing of the bone graft into fusion. The dynamic embodiment of the implant 1, 101 allows for some amount of micro-compression of the implant 1, 101 under physiologic loading, to prevent stress shielding.

8. Lack of Implant Incorporation with Vertebral Bone

The top and bottom surfaces of the implant 1, 101 according to certain embodiments of the present invention are made of titanium and are dual acid etched. The dual acid etched surface treatment of titanium allows in-growth of bone to the surfaces. Hence, the implant 1, 101 is designed to incorporate with the vertebral bone over time. It may be that the in-growth happens sooner than fusion. If so, there may be an opportunity for the patients treated with the implant 1, 101 of the present invention to return to normal activity levels sooner than currently recommended by standards of care.

9. Limitations on Radiographic Visualization

Even the titanium-only embodiment of the present invention has been designed with large windows to allow for radiographic evaluation of fusion, both through AP and lateral X-rays. The composite implant 101 minimizes the volume of titanium, and localizes it to the top and bottom surfaces and on the corners. The rest of the implant 101 is made of PEEK which is radiolucent and allows for free radiographic visualization.

10. Cost of Manufacture and Inventory

The cost to manufacture a single implant 1, 101 according to the present invention is comparable to the cost to manufacture commercially available ALIF products. But a typical implant set for a conventional device can have three foot-prints and ten heights for each foot-print. Therefore, to produce one set, the manufacturer has to make thirty different setups if the implants are machined. In contrast, for the composite embodiment according to certain embodiments of the present invention, the manufacturer will have to machine only three sets of metal plates, which is six setups. The PEEK can be injection molded between the metal plates separated by the distance dictated by the height of the implant 101. Once the injection molds are made, the subsequent cost of injection molding is considerably less as compared to machining. This feature of the present invention can lead to considerable cost savings.

In addition, a significant expense associated with a dual acid etched part is the rate of rejects due to acid leaching out to surfaces which do not need to be etched. In the case of the composite implant 101 according to certain embodiments of the present invention, the criteria for acceptance of such a part will be lower because the majority of the surfaces are covered with PEEK via injection molding after the dual acid etching process step. This feature can yield significant manufacturing-related cost savings.

Although illustrated and described above with reference to certain specific embodiments and examples, the present invention is nevertheless not intended to be limited to the details shown. Rather, various modifications may be made in the details within the scope and range of equivalents of the claims and without departing from the spirit of the invention. It is expressly intended, for example, that all ranges broadly recited in this document include within their scope all narrower ranges which fall within the broader ranges.

What is claimed:

1. A composite telescoping interbody spinal implant comprising:
a cage formed of metal, a metal alloy, or both, able to change size following manufacture, and having a top plate with a plurality of posts and a bottom plate with a corresponding plurality of columns, the posts telescopically engaging the columns upon assembly of the top plate with the bottom plate, wherein the posts extend partially outside the columns when the top plate is in a raised first position with respect to the bottom plate and the posts and columns are fully engaged when the top plate is in a second position closest to the bottom plate; and
a non-metallic body inserted between the top plate and the bottom plate and defining the adjustable height of the implant.

2. The implant according to claim 1 wherein the posts are integral with the top plate and the columns are integral with the bottom plate.

3. The implant according to claim 1 wherein, when assembled, the implant defines a vertical aperture that extends the entire height of the implant.

4. The implant according to claim 3 further comprising bone graft material disposed in an area defined by the cage including the aperture.

5. The implant according to claim 4 wherein the bone graft material is selected from cancellous autograft bone, allograft bone, demineralized bone matrix (DBM), porous synthetic bone graft substitute, bone morphogenic protein (BMP), or combinations of those materials.

6. The implant according to claim 1 wherein the columns are shaped and adapted to accommodate the impact of an instrument during placement of the implant.

7. The implant according to claim 1 wherein the cage and the body are sized, shaped, and adapted to maximize contact by the implant with the apophyseal rim of the vertebral endplates.

8. The implant according to claim 1 wherein the body has a hole facilitating manipulation of the implant.

9. The implant according to claim 1 wherein the top plate has a top surface with a roughened topography, a bottom surface which faces the bottom plate, opposing lateral sides rounded to ease placement of the implant, and opposing anterior and posterior portions with the anterior portion including a sharp edge and the posterior portion rounded to ease placement of the implant.

10. The implant according to claim 1 wherein the bottom plate has a top surface which faces the top plate, a bottom surface with a roughened topography, opposing lateral sides rounded to ease placement of the implant, and opposing anterior and posterior portions with the anterior portion including a sharp edge and the posterior portion rounded to ease placement of the implant.

11. The implant according to claim 1 wherein the top and bottom plates are each formed from two, separate sections.

12. The implant according to claim 1 further comprising at least one strut formed on the bottom plate, the top plate, or on both the top and bottom plates, the at least one strut enhancing the structural integrity of the implant and facilitating one or more of anterior, antero-lateral, and lateral implantation of the implant.

13. The implant according to claim 12 wherein the at least one strut is integral with the bottom plate, the top plate, or both the top and bottom plates.

14. The implant according to claim 12 wherein the at least one strut is formed on the bottom plate and has a height substantially equal to the height of the columns.

15. The implant according to claim 12 wherein the at least one strut has a hole facilitating manipulation of the implant.

16. A composite telescoping interbody spinal implant comprising:
a cage formed of metal, a metal alloy, or both, able to change size following manufacture, and having a top plate with a plurality of posts and a bottom plate with a corresponding plurality of columns, the posts telescopically engaging the columns upon assembly of the top plate with the bottom plate, wherein the posts extend partially outside the columns when the top plate is in a raised first position with respect to the bottom plate and the posts and columns are fully engaged when the top plate is in a second position closest to the bottom plate, wherein:
(a) the top plate has a top surface with a roughened topography, a bottom surface which faces the bottom plate, opposing lateral sides rounded to ease placement of the implant, and opposing anterior and posterior portions with the anterior portion including a sharp edge and the posterior portion rounded to ease placement of the implant, and
(b) the bottom plate has a top surface which faces the top plate, a bottom surface with a roughened topography, opposing lateral sides rounded to ease placement of the implant, and opposing anterior and posterior portions with the anterior portion including a sharp edge and the posterior portion rounded to ease placement of the implant; and
a non-metallic body inserted between the top plate and the bottom plate and defining the adjustable height of the implant,
wherein the cage and the body are sized, shaped, and adapted to maximize contact by the implant with the apophyseal rim of the vertebral endplates.

17. The implant according to claim 16 wherein the cage is titanium, a titanium alloy, or both.

18. The implant according to claim 16 wherein the body is polyetherether-ketone (PEEK).

19. The implant according to claim 16 wherein the top and bottom plates are each formed from two, separate sections.

20. The implant according to claim 16 further comprising at least one strut formed on the bottom plate, the top plate, or on both the top and bottom plates, the at least one strut enhancing the structural integrity of the implant and facilitating one or more of anterior, antero-lateral, and lateral implantation of the implant.
